(19) United States
(12) Patent Application Publication
(10) Pub. No.: US 2013/0158383 A1
(43) Pub. Date: Jun. 20, 2013

(54) BOND-SELECTIVE VIBRATIONAL PHOTOACOUSTIC IMAGING SYSTEM AND METHOD

(71) Applicants: Ji-Xin Cheng, West Lafayette, IN (US); Han-Wei Wang, West Lafayette, IN (US); Michael Sturek, Zionsville, IN (US)

(72) Inventors: Ji-Xin Cheng, West Lafayette, IN (US); Han-Wei Wang, West Lafayette, IN (US); Michael Sturek, Zionsville, IN (US)

(73) Assignee: PURDUE RESEARCH FOUNDATION, West Lafayette, IN (US)

(21) Appl. No.: 13/818,075

(22) PCT Filed: Aug. 22, 2011
(86) PCT No.: PCT/US2011/048671

§ 371 Date: Feb. 20, 2013

Related U.S. Application Data
(60) Provisional application No. 61/375,554, filed on Aug. 20, 2010.

(51) Int. Cl. A61B 5/00

(52) U.S. Cl. CPC A61B 5/0095
USPC 600/407

(57) ABSTRACT

An imaging system, including a radiation source configured to output a signal that can non-invasively and selectively cause overtone excitation of molecules based on a predetermined chemical bond, and an ultrasound detector configured to non-invasively detect an acoustic signal generated by vibrational energy caused by the selective overtone excitation of the molecules and further configured to convert the acoustic signal into an image.

### PRIORITY

The present application is related to, and claims the priority benefit of U.S. Provisional Patent Application Ser. No. 61/375,554, filed Aug. 20, 2010, the contents of which is hereby incorporated by reference in its entirety into this disclosure.

### STATEMENT REGARDING GOVERNMENT FUNDING

This invention was made with government support under EB7243 and HL062552 awarded by the National Institute of Health. The government has certain rights in the invention.

### TECHNICAL FIELD

The present disclosure generally relates to imaging systems, and in particular to an acoustic imaging system.

### BACKGROUND

Imaging tools have been essential for study of human diseases. Recently, ultrasound imaging, X-ray computed tomography, and magnetic resonance imaging (MRI) are routinely used for clinical diagnosis. Nevertheless, these techniques suffer from insufficient spatial resolution (i.e., lack of sufficient penetration into the tissue) and/or poor chemical selectivity (lack of targeting particular compounds rich in certain chemical bonds).

In biological research, optical microscopy has become an indispensible imaging tool benefiting from the development of versatile platforms and fluorescent tags, e.g., the green fluorescent proteins, and stains. However, the penetration depth of optical imaging modalities is usually limited to c.a. 100 μm, which impedes label-free detection of lesions which are positioned deeper than 100 μm.

One approach to achieve label-free chemically selective imaging is to use signals from inherent molecular vibrations. Vibrational microscopes based on spontaneous Raman scattering and infrared (IR) absorption have been widely used for chemical imaging of unstained (label-free) samples. Nevertheless, the application of IR microscopy to live cell imaging has been hindered by inadequate spatial resolution and IR absorption of water. Small cross sections of Raman scattering (i.e., weak Raman signal) also hinders tissue imaging. These limitations collectively limit the application of Raman microscopy to highly dynamic biological systems.

Another approach for producing higher image quality is the prior work of nonlinear vibrational imaging tool based on coherent anti-Stokes Raman scattering (CARS) is found in U.S. Pat. No. 6,809,814 to Xie et al. and U.S. Pat. No. 6,108,081 to Holtom et al., entirety of which are incorporated herein by reference. In a CARS process, two pulsed lasers are collinearly overlapped and tightly focused into a sample. When the frequency difference of the two lasers is in resonance with a molecular vibration, an enhanced CARS signal is produced, which provides chemical bond selectivity. Importantly, the coherent addition of CARS fields generates a large and directional signal, enabling high-speed vibrational imaging of a biological sample in a label-free manner.

Typical imaging applications include generating images of an animal's brain for visualizing the myelinated axons and cross sectional images of arteries in order to visualize lipid-laden plaques in atherosclerosis. However, because CARS microscopy has a tissue penetration depth of c.a. 100 μm, the skull of the animal would need to be opened or the tissue near the artery would need to be disturbed, resulting in highly invasive procedures. Extensive efforts have been spent to increase the penetration depth. For example, adaptive optics was shown to be able to double the penetration depth. A stick lens was employed to physically deliver the excitation beams into a thick tissue. Various nonlinear optical (NLO) microscopy strategies, including CARS, have been reported in the prior art. However, none of these strategies has significantly overcome the difficulties of small field of view and limited penetration depth.

Therefore, a label-free imaging system with chemical specificity and high spatial resolution, with sufficient penetration depth is highly desired to serve as a noninvasive imaging system or a minimally invasive imaging system that does not damage tissues during characterization of diseases in animal models and human patients.

### SUMMARY

A novel imaging system and a method associated with the system that is based on overtone excitation of molecular vibration targeting specific chemical bonds along with acoustic detection of pressure waves that are generated in a biological tissue as a result of the overtone excitation are described in the present disclosure.

According to one aspect of the present disclosure, an imaging system is disclosed. The imaging system includes a radiation source configured to output a signal that can non-invasively and selectively cause overtone excitation of molecules based on a predetermined chemical bond. The imaging system further includes an ultrasound detector configured to non-invasively detect an acoustic signal generated by vibrational energy caused by the selective overtone excitation of the molecules and further configured to convert the acoustic signal into an image.

According to another aspect of the present disclosure, a method for imaging biological tissue is disclosed. The method includes providing a radiation signal from a radiation source that can non-invasively and selectively cause overtone excitation of molecules based on a predetermined chemical bond. The method further includes receiving an acoustic signal generated by vibrational energy caused by the selective overtone excitation of the molecules. Also, the method includes converting the acoustic signal to an image representative of a biological tissue targeted by the radiation signal.

### BRIEF DESCRIPTION OF DRAWINGS

FIG. 1A depicts a block diagram of a vibrational photoacoustic (VPA) imaging system, according to the present disclosure;

FIG. 1B depicts a schematic diagram of the VPA imaging system of FIG. 1A;

FIG. 1C depicts a diagram of the 1st (2v) and 2nd (3v) overtone absorption of a molecule;

FIG. 1D depicts a graph of time vs. amplitude of a representative ultrasound waveform and the result of Hilbert transformation;

FIG. 2 depicts examples of overtone absorption ranges in wavelengths or wavenumbers for common chemical bonds found in biological matters;

FIG. 3A depicts a graph of wavelengths/wavenumbers vs. amplitude for a spectrum of the 2nd overtone absorption of CH in butanal;

FIG. 3B depicts a graph of pulse energy vs. the amplitude of the VPA signal;

FIG. 3C depicts graphs of wavelengths/wavenumbers vs. normalized amplitude of the VPA spectra for various biological compounds;

FIG. 3D depicts a graph of thickness of a collagen matrix vs. a normalized photoacoustic signal with a penetration depth of the VPA signal at about 7 mm;

FIGS. 3E, 3F, 3G, and 3H depict VPA images of a sample phantom containing oil droplet shell interrogated by using 1195 nm radiation for targeting CH rich molecules;

FIG. 4A depicts a schematic perspective view of an arterial structure with three distinct locations identified at various cross sectional depths;

FIG. 4B depicts a graph of wavelengths/wavenumbers vs. amplitude for the three locations of FIG. 4A;

FIGS. 4C and 4C′ are VPA images of maximum amplitude projection of a confluent lipid core in an atheromatous artery (FIG. 4C) and a 3-D reconstruction (FIG. 4C′);

FIGS. 4D and 4D′ are VPA images of maximum amplitude projection of a scattered lipid deposition in an arterial wall (FIG. 4D) and a 3-D reconstruction (FIG. 4D′);

FIGS. 4E and 4E′ are VPA images of maximum amplitude projection of mild fatty streaks (FIG. 4E) and a 3-D reconstruction (FIG. 4E′);

FIG. 5A depicts VPA images of maximum amplitude projection (MAP) of the intramuscular fat along the XY, YZ, and XZ planes;

FIG. 5B depicts a photomicrograph of the muscle tissue;

FIG. 5C depicts VPA spectra (i.e., wavelengths/wavenumbers vs. amplitude) of the three locations of FIG. 5A;

FIGS. 6A, 6B, and 6C depict VPA images of the lipid deposition in an atherosclerotic artery, wherein FIG. 6A depicts a C-scan image around the luminal surface, and FIGS. 6B-6C depict C-scan images at a depth over 250 μm and 500 μm from the lumen surface;

FIG. 6D depicts a 3-D reconstruction result of the VPA images which shows the lipid distribution within the arterial wall;

FIGS. 7A, 7B, and 7C depict 3-D VPA images of a malignant mammary tumor mass;

FIG. 7D depicts a 3-D reconstruction of the malignant mammary tumor mass of FIGS. 7A-7C;

FIG. 8A depicts an embodiment of an imaging device including an optical fiber for providing an overtone excitation of molecular vibration and an internal scanner for receiving the generated photoacoustic signal;

FIG. 8b depicts another embodiment of an intravascular imaging device;

FIG. 9A depicts a graph of absorption coefficient (μₛ) vs. wavelength (nm);

FIG. 9B depicts a graph of photoacoustic amplitude for various compounds (a.u.) vs. wavelength (nm);

FIG. 9C depicts VPA images of intramuscular fat using CH₂ first (FIG. 9C top panel) and second (FIG. 9C middle panel) overtone excitation;

FIG. 10A depicts a schematic of a phantom to investigate the effect of water absorption;

FIGS. 10A, 10B, and 10C depicts photoacoustic amplitude for various compounds (a.u.) vs. wavelength (nm);

FIG. 11A is a schematic of atherosclerotic artery wall as imaged by VPA microscopy with 0.5 mm thick blood layer;

FIG. 11B is a c-scan image with the 2D images at selected depths which are acquired using 1730 nm excitation;

FIGS. 11C and 11D depict VPA b-scan imaging using 1730 nm and 1210 nm excitation;

FIG. 11E depicts a VPA spectrum;

FIG. 12A shows the spectra of butter fat and rat tail tendon;

FIGS. 12B and 12C VPA depict imaging of the phantom sample performed at both 1640 nm and 1730 nm; and

FIGS. 12D-12I depict 3D VPA imaging of artery adventitia.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

A novel imaging system and a method associated with the system that is based on overtone excitation of molecular vibration targeting specific chemical bonds along with acoustic detection of pressure waves that are generated in a biological tissue as a result of the overtone excitation are described in the present disclosure. This system and the associated method provide label-free (unstained and untagged) non-invasive or minimally invasive imaging that does not damage tissues during characterization of lipid-rich atherosclerotic plaques, as well as other structures associated with various diseases. A pulsed, wavelength-tunable, monochromatic radiation is directed into a sample. The wavelength of the radiation is adjusted to match the overtone vibrational frequency of a molecule at near-infrared region. Vibrational absorption of the incident radiation and subsequent conversion of the vibrational energy into heat generates a pressure transient inside a sample, thereby producing a detectable acoustic signal having molecule-specific information.

It should be appreciated that the terms “invasive”, non-invasive, and “minimally invasive” are used interchangeably and are intended to have the same effect for the purposes of the present disclosure. Therefore, while placing an imaging probe (e.g. light and/or ultrasound) on the skin of a person would be “non-invasive”, arterial or venous placement of the same probe is “minimally invasive.”

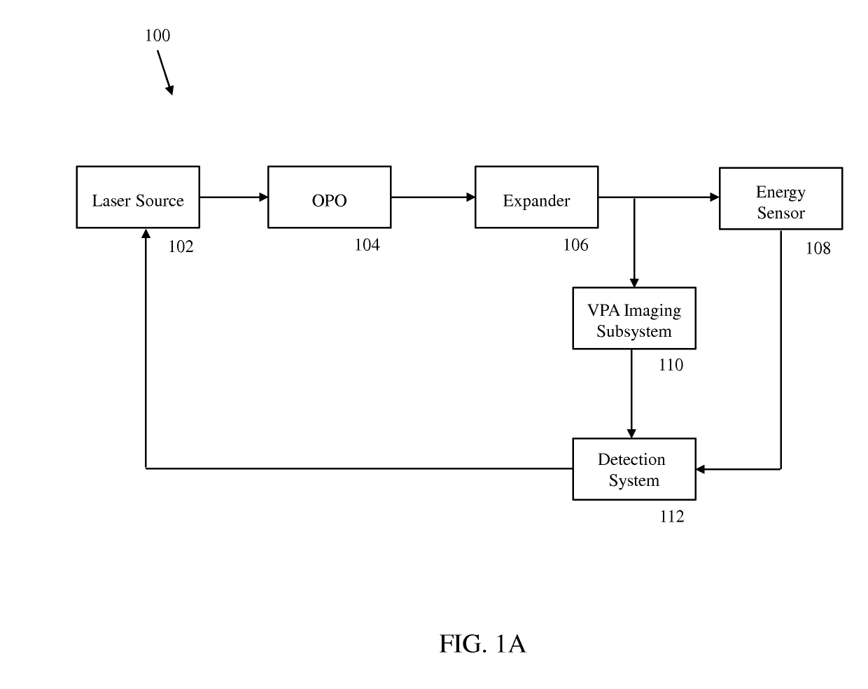

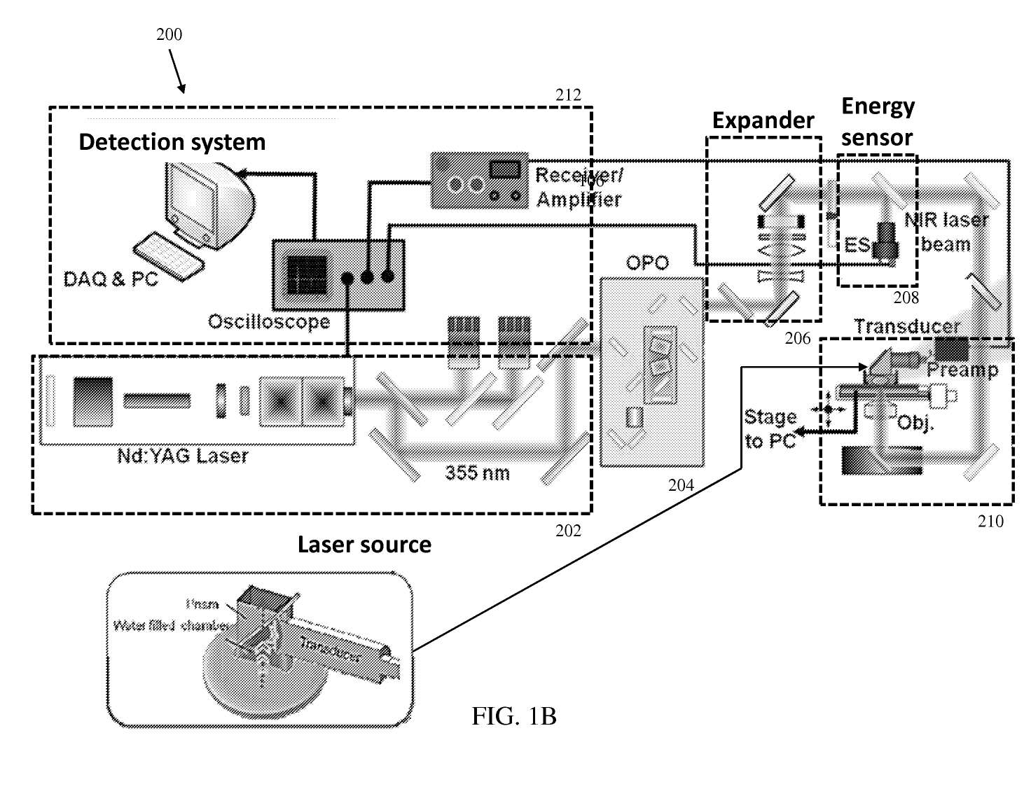

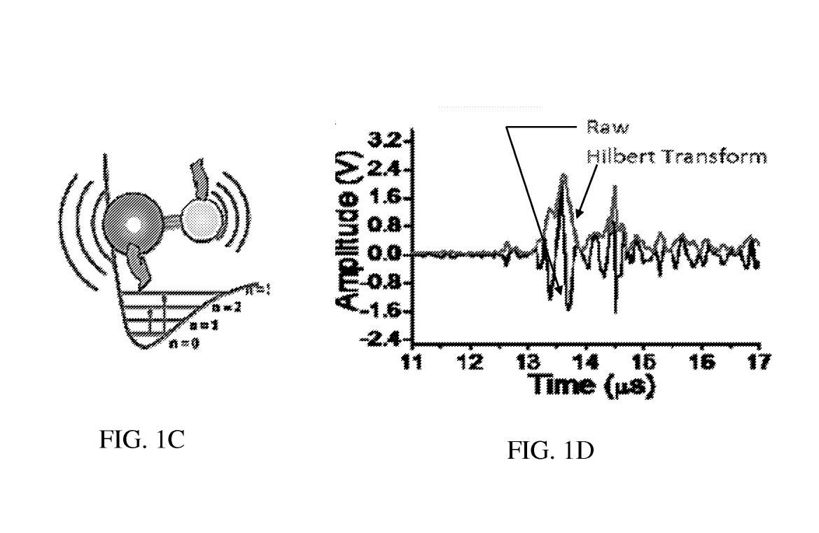

Referring to FIG. 1A, a block diagram of a vibrational photoacoustic (VPA) imaging system 100, according to the present disclosure. The system 100 includes a laser source 102 which provides an optical radiation source to an optical parametric oscillator (OPO) 104. The OPO 104 provides a near infrared to an expander 106. The expander 106 employs a doublet lens setup (f=30 mm) to weakly focus the beam on a microscope platform, represented as the VPA imaging subsystem 110. The output of the expander 106 is also provided to an energy sensor 108. Both the energy sensor 108 and the VPA imaging subsystem 110 communicate with a detection system 112 which provides a feedback control signal to the laser source 102.

Referring to FIG. 1B, a schematic diagram of a VPA imaging system 200, according to the present disclosure, is depicted. Similar to the block diagram depicted in FIG. 1A, the system 200 includes a laser source 202 which provides an optical radiation source to an optical OPO 204. The OPO 204 provides a near infrared (NIR) to an expander 206. The expander 206 weakly focuses the NIR beam on a microscope platform, represented as the VPA imaging subsystem 210. The output of the expander 206 is also provided to an energy sensor 208. Both the energy sensor 208 and the VPA imaging subsystem 210 communicate with a detection system 212 which provides a feedback control signal to the laser source 202.

The VPA imaging subsystem 210 is provided on an inverted microscope platform for detecting and recording generated ultrasound signals. The photoacoustic transients are recorded by data acquisition devices via commercially available data acquisition package. A Hilbert transformation is performed, as further described below with respect to FIG. 1D, to retrieve the envelope of signal amplitude for further signal analysis and image reconstruction.

To explore photoacoustic imaging based on overtone absorption of molecules as the contrast mechanism, 5-nanosecond pulse trains were used in the near-infrared region generated by an Nd:YAG pumped optical parametric oscillator (OPO) laser system (i.e., the laser source 202 and the OPO 204). The idler output from the OPO 204 is tunable from 740 nm to 2400 nm covering overtone absorption wavelengths of interest. Instead of using tightly focused beam(s) as in nonlinear vibrational microscopy, the system demonstrated here employs the expander 206 which uses doublet lens (f=30 mm) to weakly focus the beam on the microscope platform. The focal volume, which determines the lateral resolution, is in a confocal geometry related to the focus of an ultrasound transducer used to detect photoacoustic pressure transients. The focused-type transducer has a center frequency of 20 MHz with a 50% bandwidth that theoretically gives an axial resolution of about 132 μm. Ultrasound transients are detected through an ultrasound splitter and recorded via a preamplifier which is part of the VPA imaging subsystem 210 and a signal receiver/amplifier which is part of the detection system 212.

The pertinent laser radiation is aligned into the inverted microscope platform of the VPA imaging subsystem 210. An objective lens is used to weakly focus the radiation pulses into a sample to induce a photoacoustic effect at various planar locations. The generated acoustic signal is detected by a transducer (depicted in FIG. 1B as an exploded view) and recorded through data acquisition devices (which are part of the detection system 212), as shown in FIG. 1B. The wavelength of incident radiation is selected according to the overtone absorption of molecules of interest and chemical bonds within those molecules.

### The Photoacoustic Effect

A photoacoustic effect takes place when radiation is absorbed by a tissue sample. The absorbed energy is converted to heat which then causes local thermal expansion through the thermal elastic process. The thermal expansion thereafter generates pressure wave transient that propagates through the sample tissue as an acoustic wave and can be detected by one or more transducers. Information obtained from the amplitude and the time-of-flight of the acoustic waves can be used to construct an image of the absorbing structure of tissues. Different biological tissues have different photoacoustic responses because of differences in absorption coefficient, thermal elasticity, size of absorber, etc. It should also be appreciated that different acoustic waves initiated by different structures arrive at the transducers at different times. This is because of flight times of these waves differ based on the depths of the structures, as the ultrasound waves propagate at the speed of sound within a tissue. The photoacoustic signal has been used for mapping vessel plexuses benefiting from the strong contrast from electronic absorption of hemoglobin in the visible region. Oxygenated and deoxygenated blood can be distinguished. Other than hemoglobin, image contrasts, strains, or labels, such as dyes and nanoparticles are used as contrast agents for probing specific targets. Photoacoustic imaging is disclosed in U.S. Pat. App. No. 20050070803, published on Mar. 31, 2005, and U.S. Pat. App. No. 20050004458, published on Jan. 6, 2005, entirety of which are incorporated herein by reference.

According to one embodiment of the current disclosure, a tunable nanosecond (ns) laser is used to induce overtone vibration absorption of selected molecules and more particularly, molecules with selected chemical bonds. The wavelength is typically in the near infrared region depending on the vibrational band of interest. The generated ultrasound waves is detected by a transducer and recorded through amplifier(s) and custom built data acquisition devices.

Overtone absorption is an important principle of near-infrared spectroscopy that measures bulk absorbance or reflectance of samples. According to the anharmonicity theory, the frequency of an overtone band is described by v̅= v̅₁n−χ v̅₁(n+n²) where is the frequency of the fundamental vibration, χ is the anharmonicity, and n=2, 3, . . . represent the first, second, and so on, overtones.

Referring to FIG. 1C, a diagram representation of overtone excitation is depicted. Using the near-infrared spectroscopic approach, molecular spectra in chemical and biological samples can be excited according to radiation signals representing the overall overtone absorption and the elastic scattering in a sample. The spectral information can also be retrieved to perform a molecular scan or chemogram of biological tissues, e.g. atherosclerotic arteries. The bulk measurement of absorbance or reflectance, however, obscures depth information. The elastic scattering further compromises the imaging potential of near-infrared spectroscopy. Notably, most of the second overtone frequencies of molecules of interest are located in the near-infrared region from 700 to 1300 nm, where the background tissue is minimally absorbing. Within this spectral region, overtone vibrational absorption provides opportunities to generate a chemically selective photoacoustic transient in a biological structure.

FIG. 1D depicts a graph of time vs. amplitude of a representative ultrasound waveform and the result of the Hilbert transformation.

As illustrated in the table below, three exemplary chemical bonds can be excited using corresponding radiation frequencies.

TABLE 1
Bond-specific excitation wavelengths

| | Chemical bond to be | Molecule to be |
|---|---|---|
| Excitation wavelengths | excited | mapped |
| 1150 to 1240 nm | CH bond, second | Lipids |
| | overtone | |
| 1430 to 1500 nm | OH bond, first | Water |
| | overtone | |
| 950 to 1030 nm | NH bond, second | Proteins |
| | overtone | |

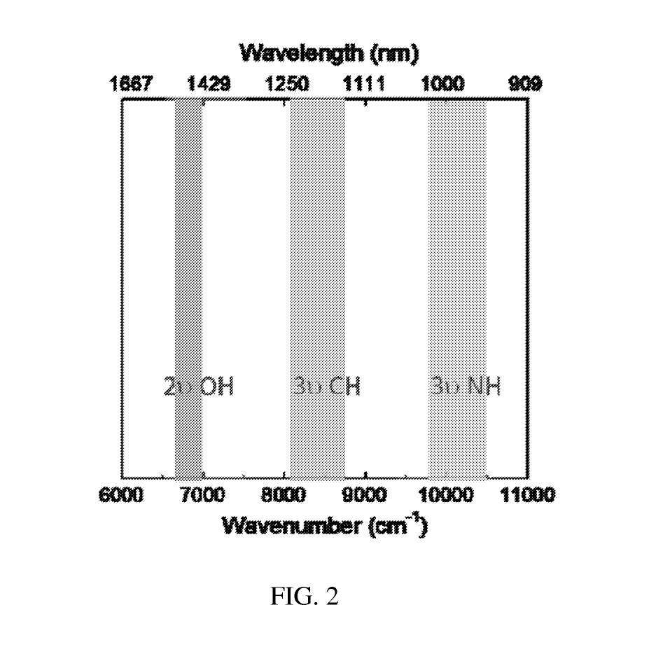

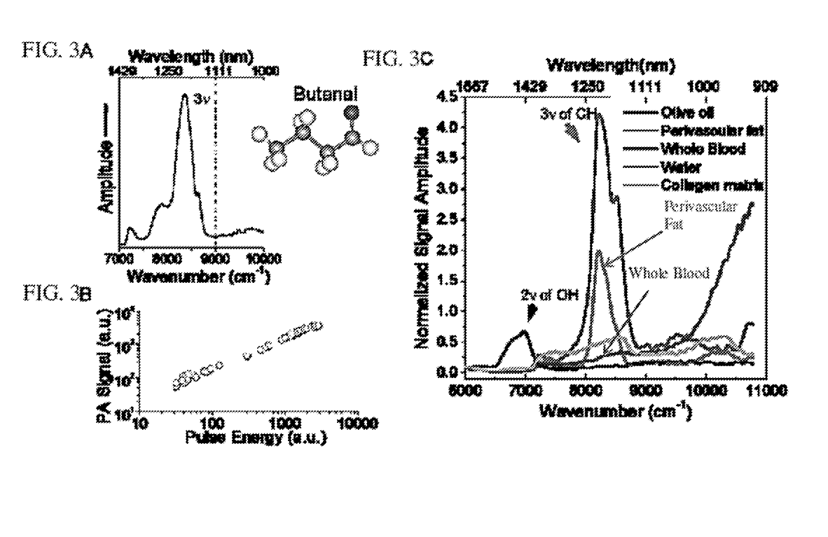

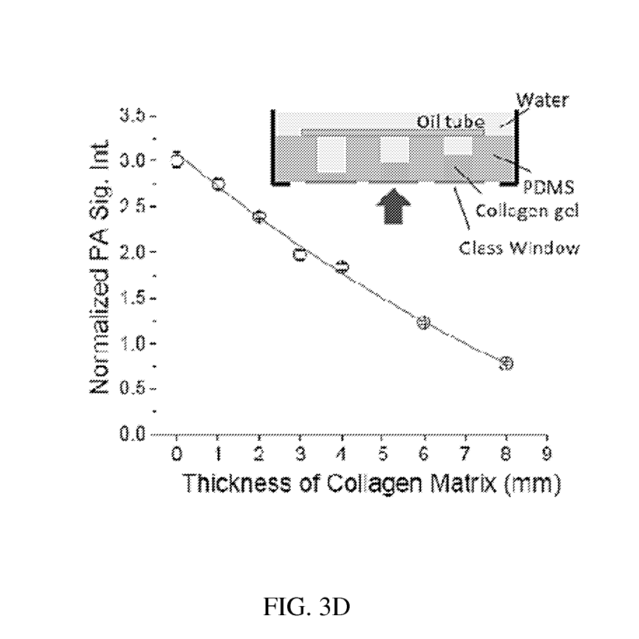

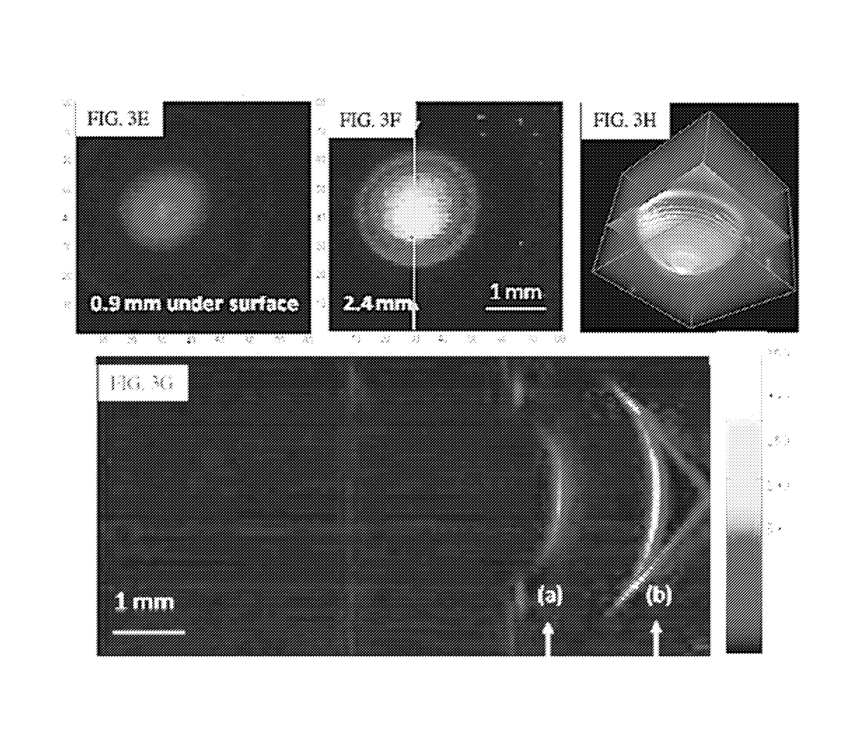

Referring to FIG. 2, a graph of wavenumbers corresponding to the common chemical bonds found in biological matters and provided in the table above is depicted. The graph shown in FIG. 2 can be helpful in tuning the radiation source to generate bond-specific excitation. For example, to generate the photoacoustic signal from overtone excitation of CH bond, butanal, a CH-rich liquid, was loaded in a glass tube in which the sample volume and location were controlled. Referring to FIG. 3A, a graph of wavelength vs. amplitude for a spectrum of the 2nd overtone absorption of CH in butanal is depicted. The wavenumber peak is around 8400 cm-1, corresponding to a wavelength of 1190 nm. Referring to FIG. 3B, a graph of pulse energy vs. the amplitude of the VPA signal is depicted. The VPA signal is found to be linearly proportional to the energy of radiation pulses (FIG. 3B).

Applying the VPA spectroscopy to biologically significant samples, the spectroscopic results show that CH-rich samples produce a strong VPA signal around 1200 nm due to the second overtone absorption of CH vibration. Referring to FIG. 3C graphs of wavenumbers vs. normalized amplitude of the VPA spectra for various compounds are depicted. Specifically, at a wavelength of 1215 nm, the VPA signal from adipose tissues is over 7 times higher than that from blood and over 5 times higher than that from collagen. In addition, the VPA signal from the first overtone absorption of OH is located around a wavelength of 1400 nm (i.e., wavenumbers of 6500 to 7500 cm⁻¹), and the signal from the second overtone absorption of NH is detectable around a wavelength of 950 nm. As a result, and as depicted in FIG. 3C, a signal of CH 2nd overtone form lipids is able to be distinguished from that of whole blood or water at a wavenumber of about 8300 cm⁻¹.

To further demonstrate the efficacy of the VPA imaging according to these teachings, VPA imaging in a collagen matrix was studied. FIG. 3D depicts a graph of thickness of a collagen matrix vs. a normalized VPA signal showing depth of the VPA signal is about 7 mm at the e-1 signal level in the semi-opaque collagen-matrix phantom.

FIGS. 3E, 3F, 3G, and 3H demonstrate 3D vibrational photoacoustic imaging of a tissue phantom containing an oil bubble shell, interrogated by using 1195 nm radiation for targeting CH rich molecules. FIGS. 3E-3G show reconstruction images of sections along lateral and axial directions. FIG. 3H shows a 3-D reconstruction of an oil droplet shell inside the phantom. It should be appreciated that the lipid deposition in an atheromatous arterial wall can be imaged with this method from the artery's luminal side.

### Applications of VPA Imaging

For biomedical applications, 3-D VPA imaging of lipid-rich atherosclerotic plaques optically excited from the lumen side have been performed. Lipid deposition is a major hallmark in atherosclerosis that predominates the lesion progression and plaque vulnerability to rupture. Monitoring the lipid content in an arterial wall is one important factor for vascular intervention in diagnosis and treatment of atherosclerosis. To demonstrate label-free VPA imaging of atherosclerotic lipid depositions, carotid arteries were harvested from Ossabaw pigs having metabolic syndrome and profound atherosclerosis. Spectroscopic analysis and 3-D imaging were conducted from the luminal side of the artery. Referring to FIG. 4A, a schematic perspective view of an arterial structure with three distinct locations identified at various cross sectional depths is depicted. VPA spectroscopy at different sites of atheromatous arterial walls demonstrated the capability of sensing different levels of lipid accumulation. Referring to FIG. 4B, a graph of wavenumbers vs. amplitude for the locations of FIG. 4A is provided. Locations I, II, and III in FIG. 4A correspond to a thickened intima, an intermediate plaque without a necrotic core or fibrotic lesion, and a relatively advanced lesion with the formation of a lipid core, respectively. According to the VPA spectra of the lipid depositions in atheromatous arterial walls, radiation at 1195 nm for 3-D VPA imaging of atherosclerotic lipid deposition with optimal vibrational contrast from the lipid depositions was used. The images reveal different milieus of lipid accumulation in arterial walls such as a confluent lipid core in an atheromatous artery (FIG. 4C), a scattered lipid deposition in an arterial wall (FIG. 4D), and the formation of mild fatty streaks in early atheroma (FIG. 4E). Therefore, FIGS. 4C and 4C′ are VPA images of maximum amplitude projection of a confluent lipid core in an atheromatous artery (FIG. 4C) and the associated 3-D reconstruction (FIG. 4C′). FIGS. 4D and 4D′ are VPA images of maximum amplitude projection of a scattered lipid deposition in an arterial wall (FIG. 4D) and the associated 3-D reconstruction (FIG. 4D′). FIGS. 4E and 4E′ are VPA images of maximum amplitude projection of mild fatty streaks (FIG. 4E) and the associated 3-D reconstruction (FIG. 4E′).

A strong VPA signal from lipids located at 1.5 mm below the lumen was detectable. The VPA method that enables 3-D imaging could be a significant improvement over the existing near-infrared method.

As another application of VPA microscopy, the intramuscular fat in a fresh muscle tissue was examined. Referring to FIG. 5A, VPA images of maximum amplitude projection (MAP) of the intramuscular fat along the XY, YZ, and XZ planes are depicted including three locations (I, II, and III) identified in FIG. 5A in particular. Intramuscular lipids are involved in metabolic disorders but the assessment in fresh tissues is difficult. The intramuscular lipid may be visible by the naked eye. For example, referring to FIG. 5B, a photomicrograph of the muscle tissue is depicted. These images are typically assessed by marble score or measured chemically. With a penetration depth of over 1 mm, the 3-D VPA image of intramuscular fat, e.g., VPA images of FIG. 5A, inspected at the overtone absorption of CH around 1200 nm shows the potential of using VPA microscopy for quantitative measurement of intramuscular fat accumulation in metabolic disorders. For example, referring to FIG. 5C, VPA spectra of the three locations marked in FIG. 5A are depicted.

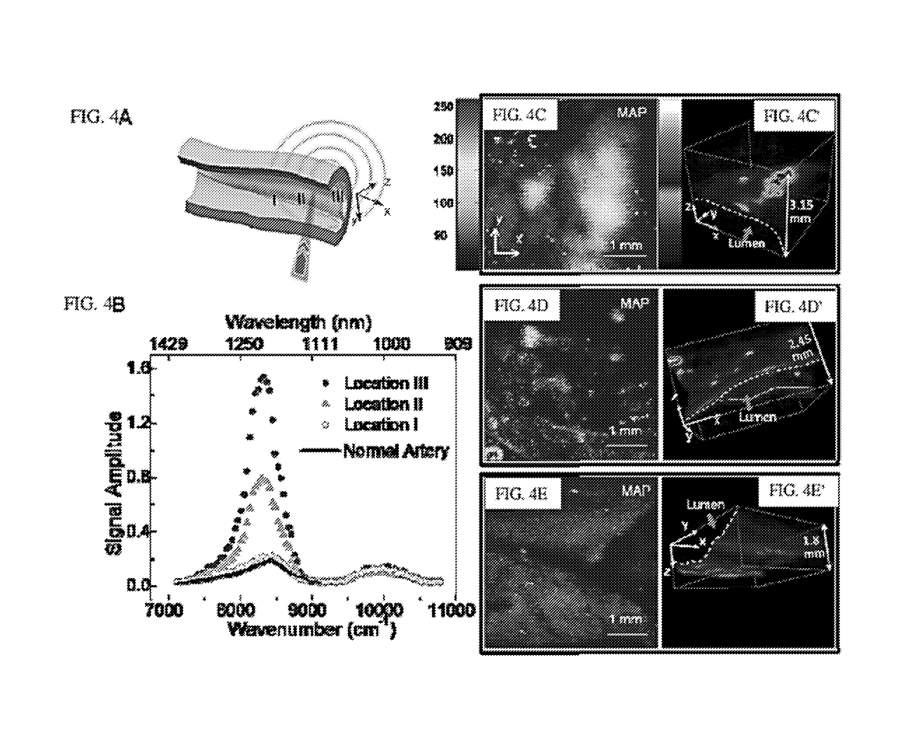

FIGS. 6A, 6B, and 6C depict C-scan images around the luminal surface and at a depth over 250 μm, and 500 μm from the lumen surface, respectively. These figures show VPA images that identify the lipids deposited in an artery. The result exemplify the significant potential of the proposed imaging system and method for biomedical applications, particularly regarding the advantages of label-free bond-selectivity and the nature of deep tissue penetration of the photoacoustic imaging. A 3-D reconstruction of the VPA image is depicted in FIG. 6D which shows the lipid distribution within the arterial wall. The green portion indicates the lipid deposition under the lumen.

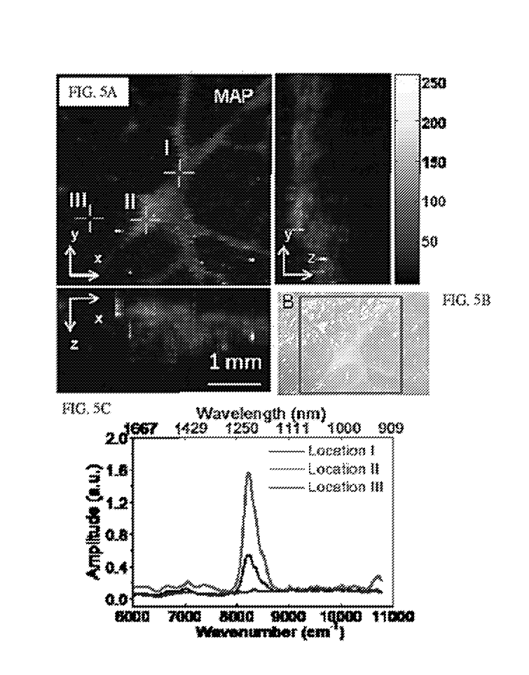

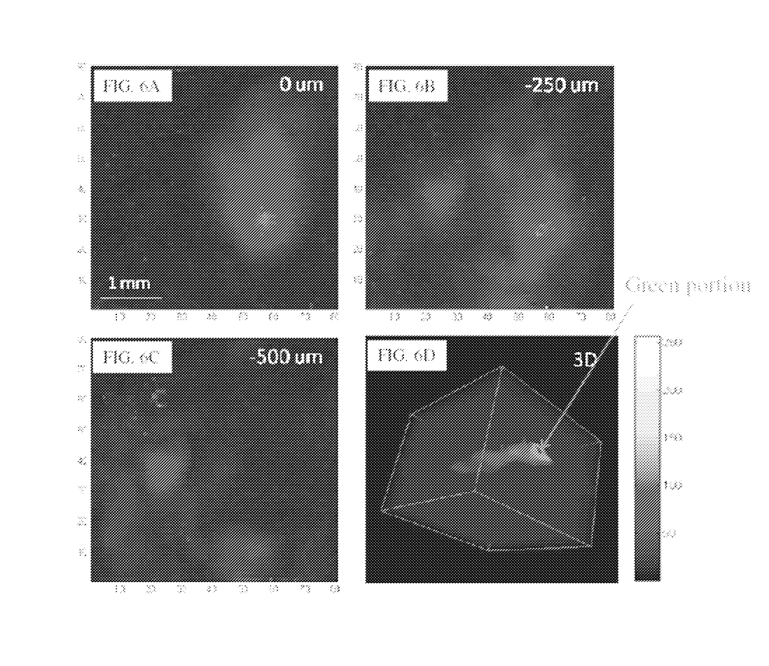

Another application of the VPA is diagnosing mammary tumor mass. The mammary lipid distribution can be mapped using the VPA imaging system. Referring to FIGS. 7A, 7B, and 7C, 3-D VPA images of a malignant mammary tumor mass are depicted. Therefore, the system described herein is additionally advantageous in detecting the location of a mammary tumor relying on the environmental changes.

Furthermore, detecting diseases in skin is another important application of the VPA system of the current disclosure. Skin plays an important role in human physiology by providing a protective barrier against germs, an insulation layer against fluctuating temperatures, and a sensory organ for heat, touch, and pain. Skin includes three main layers: an epidermis outer layer with melanocytes, a dermis second layer with nerve endings, sweat glands, sebaceous glands, and hair follicles, and a third fatty layer of subcutaneous tissues. While the skin conditions and diseases are vast, the widely known include melanoma, acne, and hair loss. Skin is highly accessible to optical examination by being a superficial structure. Comprising water and lipid-rich structures, including the sebaceous glands and adipocytes, skin is an ideal target for VPA imaging.

Also, detection of myelin loss in central and peripheral nerve system is yet another application suitable for the VPA system of the present disclosure. Demyelination, or the loss of the myelin sheaths around axons, is a hallmark of many neurodegenerative diseases such as leukodystrophies and multiple sclerosis. The loss of the myelin sheaths impairs signal conduction along axons and reduces the communication among nerve cells. The myelin membranes contain about 70% lipids by weight, and the high-density CH2 groups is expected to produce a large VPA signal.

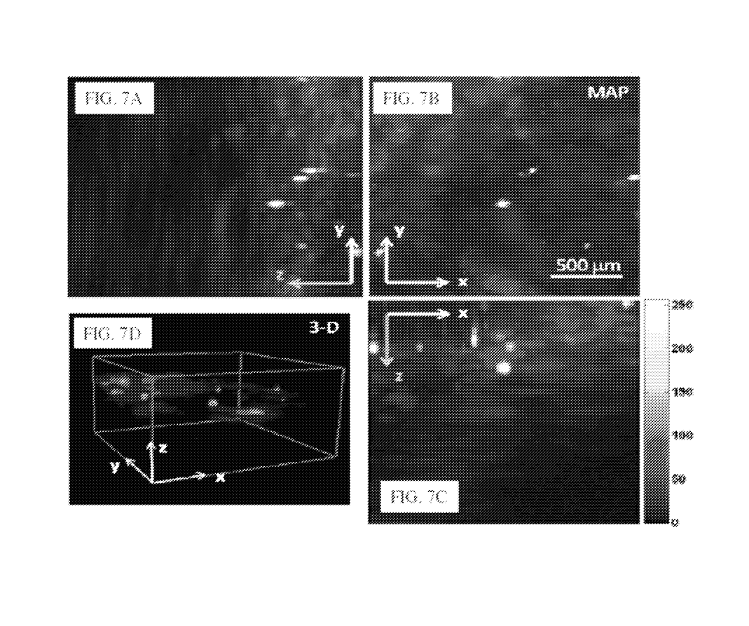

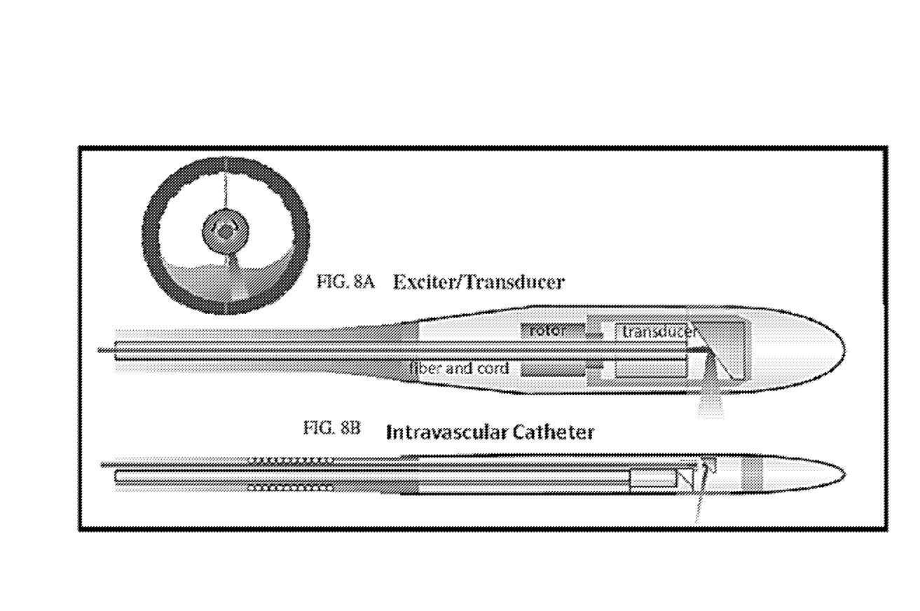

FIG. 8A depicts a schematic drawing of an embodiment of a catheter that can be used with the VPA imaging system of FIGS. 1B and 2A. The catheter is an intravascular device including an internal scanning mechanism for performing the VPA imaging. Radiation for generating the photoacoustic signal is delivered by a pertinent optical fiber. Signal is received by a miniaturized ultrasound transducer for image reconstruction. FIG. 8B depicts a schematic of an alternative embodiment of a catheter that can be used with the VPA system of FIGS. 1B and 2A with an external scanning mechanism for performing imaging. The scheme combines the configuration used in current intravascular ultrasound imaging and the requirement for VPA imaging. Signal is generated by the radiation delivered through a fiber, which is attached to the transducer and rotated simultaneously. Reconstructed B-scan image allows the identification of plaque components in arteries. These catheter devices will permit intravascular VPA (IVPA) imaging in living animals and humans.

### Imaging of Deep Tissue Through the Optical Window Between 1.6 and 1.85 μm

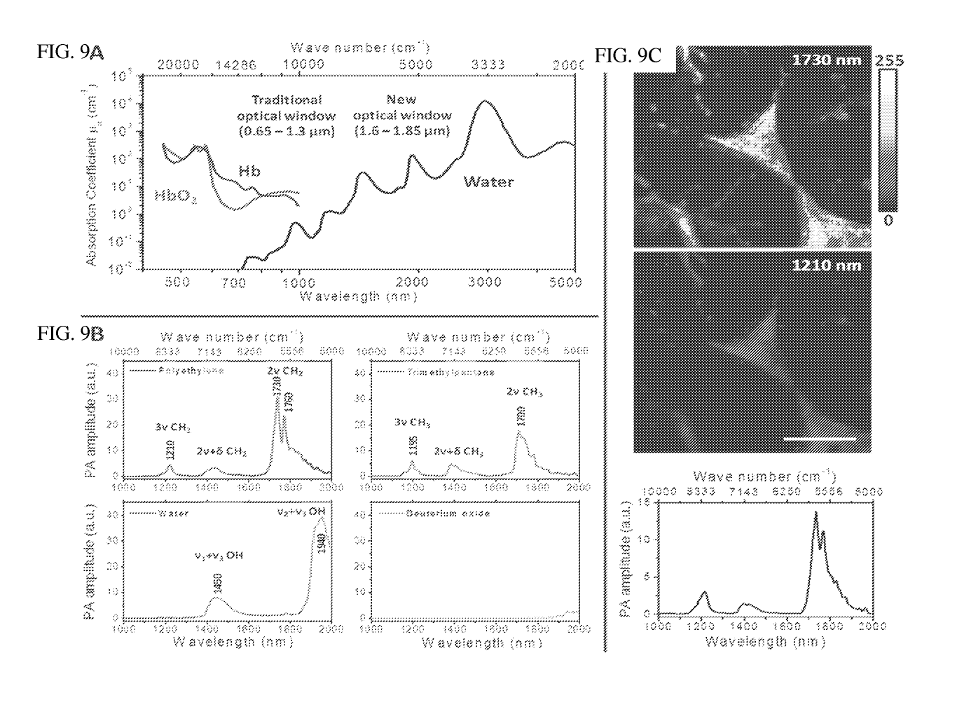

Until now, the consensus is that the gold optical window lies between 0.65 and 1.4 μm. It is commonly believed that the window stops at 1400 nm due to significant water absorption at longer wavelengths. Nevertheless, we have realized that the water absorption between 1.0 and 3.0 micron is modulated by the vibration transition of H₂O, namely the fundamental symmetric vibration v₁ and asymmetric vibration v₃ at 2900 nm, v₂ (bending)+v₃ at 1938 nm, v₁+V₃ at 1453 nm, second combinational transition at 1200 μm, and second overtone transition at 979 nm. FIG. 9A depicts a graph of absorption coefficient (cm⁻¹) vs. wavelength (nm). It should be noted that a valley exists between 1.6 and 1.85 μm, where the absorption coefficient of pure water is at the same level as that of heme proteins in whole blood around 800 nm. Considering the reduced scattering and diminished phototoxicity at longer wavelength excitation, the new optical window from 1.6 to 1.85 μm is appealing for deep tissue imaging. Importantly the first overtone of CH vibration, which has higher transition strength by one order of magnitude compared to the second overtone, is located at the same window of 1.6 to 1.85 μm. Such spectral features are advantageous in performing label-free imaging by first overtone excitation and acoustic detection. In this disclosure photoacoustic imaging of arterial plaques are provided by excitation of the first overtone of CH bond at 1.73 μm from the lumen through a layer of whole blood.

In order to identify the valid contrast in the new window, the VPA spectra of major functional groups were recorded. FIG. 9B shows the VPA spectra of polyethylene, trimethylpetane, water and deuterium oxide. The spectrum of polyethylene provides the absorption profile of the methylene group (CH₂). The CH₂ first overtone (2v CH₂) region has two primary peaks at around 1730 nm (5800 cm⁻¹) and 1760 nm (5680 cm⁻¹). The stronger peak at 1730 nm is generally thought to be a combination band of asymmetric and symmetric stretching (v₁+v₃). The 1760 nm peak is assigned to the first overtone of the asymmetric stretching or the symmetric stretching. The second combination of CH₂, located between 1.35 and 1.50 μm, is attributed to the combination of harmonic stretching and non-stretching, such as bending, twisting and rocking (2v+δ). The CH₂ second overtone region has the peak around 1210 nm. Noticeably, the VPA amplitude at 1730 nm is around 6.3 times higher than that at 1210 nm for the polyethylene sample.

The spectrum of trimethylpentane is mainly contributed by the absorption profile of methyl group (CH₃). The primary peak at around 1700 nm (5880 cm⁻¹) is assigned to the first overtone of CH₃ stretching. Two separate peaks at 1695 nm and 1704 nm, which are attributed to first overtone of asymmetric and symmetric CH₃ stretching, can be distinguished if high spectral resolution is applied. It is a remarkable fact that the CH₂ and CH₃ groups have distinguishable profiles at the first overtone region. The second combination band of CH₃ starts from 1350 nm to 1500 nm with the main peak at around 1380 nm, which is generally thought to be 2v+δ. The CH₃ second overtone has the primary peak at around 1195 nm.

In the water spectrum, the band at around 1450 nm is generally referred to as first overtone of OH stretching, however, it is actually a combination band of O-H asymmetric and symmetric stretching (v₁+v₃). The peak around 1940 nm is assigned to combination of bending and asymmetric stretching of water molecules (v₂+v₃). Excitingly, no major water absorption peak is found in between the two primary water combination absorption bands, where the strong CH₂ and CH₃ first overtone regions are located. Therefore, a potential optical window for deep-tissue CH bond imaging can be created at the water absorption ‘valley’ at around 1600-1850 nm region. In addition, no significant absorption peak is found in the wavelength range lower than 1900 nm, which indicates that deuterium oxide can be an ideal VPA coupling medium between excitation light and samples for VPA imaging and spectral measurements.

### VPA Imaging of Intramuscular Fat Based on the First and Second Overtone Transition of C—H

Since first overtone absorption coefficient is higher than that of second overtone, more photoacoustic signal should be produced with first overtone excitation, which leads to contrast enhancement in VPA imaging. FIG. 9C shows the VPA images of intramuscular fat using CH₂ first (FIG. 9C top panel) and second (FIG. 9C middle panel) overtone excitation. Those two images are maximum amplitude projection (MAP) from the same gated region (80 ns). When the same pulse energy (45 μJ) is applied for both 1730 nm and 1210 nm beam, 5 times contrast enhancement is demonstrated when using CH₂ first overtone excitation (1730 nm). As we noticed in the experiment, 45 μJ at 1210 nm is very close to the tissue damage threshold and a small amount of tissue burning is observed. On the contrary, no tissue damage is observed when using 1730 nm excitation. The tissue damage threshold is improved when using longer wavelength, possibly because negligible linear or nonlinear electronic absorption occurs when using 1730 nm excitation while 1210 nm pulse laser can still induce sufficient amount of two-photon electronic absorption. To confirm that the contrast comes from the CH₂ vibrational bands, VPA spectrum is taken at the selected position (cross in FIG. 9C top panel) where the high fat accumulation is expected. As seen in the FIG. 1C bottom panel, two primary peaks at around 1730 nm and 1760 nm within first overtone region are observed and the whole spectra highly correlate with the CH₂ absorption profile. As the higher contrast and improved damage threshold are demonstrated, a 3 dimensional (3D) intramuscular fat mapping is performed. It can be seen that a lipid network formed by intramuscular fat, which suggests that 3.5 mm tissue penetration can be reached. As we observed, the intramuscular fat network does not form a line shape structure inside the muscle tissue, but rather “dotted” or “dashed” lines. This phenomenon is possibly the result of “shadow effect”. This can be explained by making the observation that the upper fat absorption attenuates the energy reaching to deeper fat content, which affects the signal from the deeper fat content.

### Effect of Tissue Absorption and Scattering at 1730 Nm and 1210 Nm

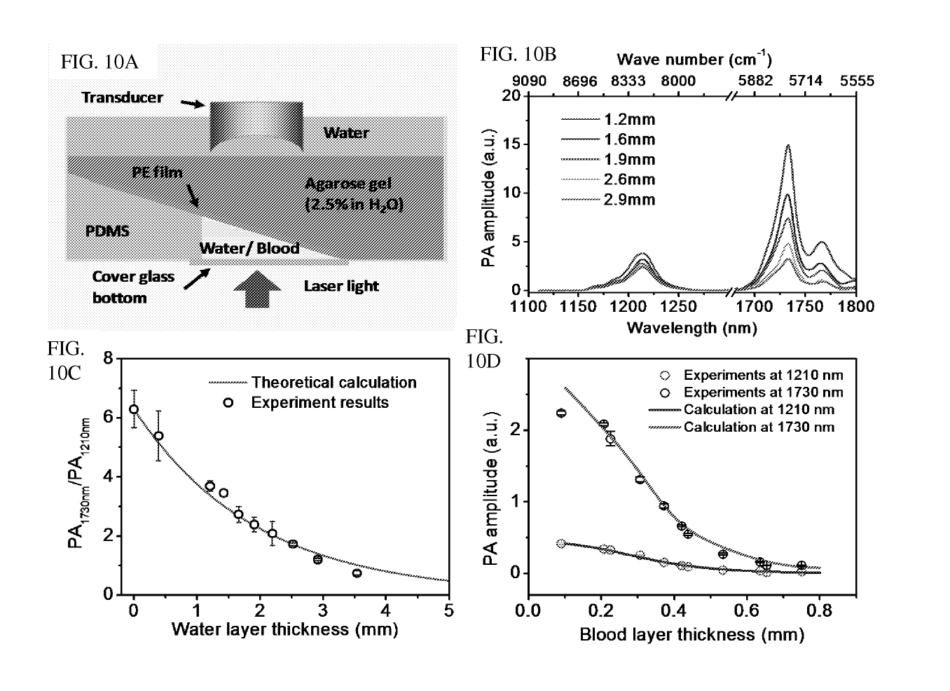

Although there is a local minimum at the water absorption spectra, the water absorption at 1730 nm is around 5 times larger than that at 1210 nm. As biological tissue consists of a large amount of water, it is important to evaluate the effect of water absorption to the CH group first overtone and second overtone excitation. In order to investigate the effect of water absorption, a phantom was constructed as shown in FIG. 10A. A PDMS wedged well was created in a cover glass bottom dish. Water was added into the well and covered by a polyethylene film which served as the signal origin. The polyethylene film was then covered with 2.5% agarose-water gel. When moving the sample from right to left while scanning the excitation wavelengths, the PA spectra of polyethylene at different water thickness can be obtained (see FIG. 10B). In general, the transient pressure generated from photoacoustic effect p can be estimated by

$$p = ( \frac{β {c}^{2}}{{c}_{p}} ) {μ}_{a} I = Γ {μ}_{a} I \qquad ( 1 )$$

Where β is the isobaric volume expansion coefficient in K⁻¹, c is the speed of sound, Cₚ is the specific heat in J/(K kg), μₐ is the absorption coefficient in cm⁻¹, I is the local light fluence in J/cm², Γ is referred to as the Grüneisen coefficient expressed as Γ=βc²/Cₚ. Since the local light fluence attenuation by water absorption follows the Beer-Lambert law, the signal generated from polyethylene through a layer of water can be expressed by

p(z)=Γμ_{a(PE)}I₀e^{-μ}^{_{a(water)}}^{z} (2)

Where z is the thickness of the water, Iₒ is the incident light fluence, and μ_{a(PE)} and μ_{a(water)} are the absorption coefficients of the polyethylene sample and water, respectively. Since the polyethylene absorption at 1730 nm is estimated to be 6.3 times larger than that at 1210 nm, the ration between photoacoustic signal at 1730 nm and PA signal at 1210 nm (PA₁₇₃₀ₙₘ/PA₁₂₁₀ₙₘ) as function of water thickness can be expressed by

$$\begin{matrix}\frac{{PA}_{1730}}{{PA}_{1210}} ( z ) = \frac{Γ {μ}_{a ( PE , 1730 )} {I}_{o} {s}^{- {μ}_{{a ( water , 1730 )}^{z}}}}{Γ {μ}_{a ( PE , 1210 )} {I}_{o} {s}^{- {μ}_{{a ( water , 1210 )}^{z}}}} \\ = 6.3 \times {e}^{- {( {μ}_{a ( water , 1730 ) -} {μ}_{a ( water , 1210 )} )}^{z}}\end{matrix} \qquad ( 3 )$$

Considering the water absorption at 1730 nm and 1210 nm, which are 6.40 cm⁻¹ and 1.26 cm⁻¹, respectively, the equation 3 can be graphed in FIG. 10C (solid line). Combining the experiment results (round dots in FIG. 10C), it is indicated that we can still benefit from 1730 nm excitation through around 3 mm thick of water layer.

Scattering is another critical factor which affects the PA signal in real tissue. The optical path for a photon to reach a certain depth increases, when more scattering events occur, thus increases the possibility of a photon to be absorbed. In the NIR region, the tissue scattering can be described approximately using Mie scattering theory. As the light wavelength increase, the scattering effect reduces. It means that using longer wavelength at 1730 nm has advantage in reducing scattering effect, thus leads to higher excitation light deliver efficiency.

As a special case, whole blood has very large scattering coefficient 40. This means that whole blood should significantly benefit from longer wavelength in photoacoustic imaging through blood. It is crucial to investigate this scenario since intravascular optical imaging suffers from huge blood scattering. With the phantom construction, shown in FIG. 10A, water was changed to rat whole blood in the wedged well. The photoacoustic signal was measured from polyethylene with both 1730 nm excitation and 1210 nm excitation as function of blood layer thickness. Both of the results are then normalized to the photoacoustic signal acquired when using 1210 nm excitation without blood layer (round hollow dots in FIG. 10D). The light delivery efficiency using Monte Carlo (MC) simulation was also estimated (see further details discussed below). The light power which is delivered to transducer focused area is normalized by the light power incident. The result is then multiplied by the factor that is induced by different polyethylene absorption coefficient at 1730 nm and 1210 nm (6.3 for 1730 nm and 1 for 1210 nm). As can be seen at FIG. 10D, the experiment results match the calculation based on MC simulation. This result indicates that using 1730 nm excitation helps gain 5-6 times when less than 1 mm blood layer presents compared to 1210 nm excitation, owing to both higher absorption coefficient in first overtone region and lower scattering effect at longer wavelength.

### 3D VPA Imaging of Atherosclerotic Artery Wall in the Presence of Whole Blood

Imaging lipid deposition inside the artery wall is a crucial topic in atherosclerosis diagnosis. Many advanced techniques have been developed to characterize the atherosclerotic plaque, including multidetector spiral computed tomography (MDCT), magnetic resonance imaging (MRI), intravascular ultrasound (IVUS), optical coherent tomography (OCT) and intravascular near infrared (NIR) spectroscopy. However, those techniques have limitations in either lack of chemical selectivity or a substantial distortion by blood when performing in vivo catheter-based imaging. VPA imaging using 1200 nm excitation is shown to be applicable in lipid mapping inside artery wall, however, it is also shown that the contrast would be diminished if a significant amount blood layer is presented (see FIG. 10C). Applying longer wavelength at CH₂ first overtone region is a feasible solution due to the benefit from both enhancement of contrast and reduction of scattering effect as demonstrated previously.

To demonstrate this, atherosclerotic artery wall is imaged by VPA microscopy with 0.5 mm thick blood layer (FIG. 11A). The atherosclerotic illac artery sample is extracted from an Ossabaw pig which was fed with atherogenic diet. As shown in FIG. 11A, the artery sample is cut open longitudinally and placed in the sample container. Between the sample and excitation light, there is a 0.5 mm thick whole blood layer extracted from adult Sprague Dawley rat. A focused ultrasound transducer is placed at the opposite side from the excitation. The 3D c-scan image with the 2D images at selected depths which are acquired using 1730 nm excitation is shown in FIG. 11B. It is indicated that a lipid core which is around 1 mm deep under the lumen is observed, and several scattered lipid depositions are detected near the lumen surface as well. Surprisingly, the blood layer also gives a strong contrast. The reason is that the blood layer is close to the excitation and attenuates the energy reaching to the artery sample. Fortunately, the artery sample and blood layer can be well differentiated owing to the depth resolvability of photoacoustic technique. One thing that needs to be mentioned is that the blood is sandwiched by two cover glasses. As the result, the ultrasound signal from the blood layer is reflected by the glasses for multiple times, leaving the layered-like signal.

The comparison between 1730 nm and 1210 nm excitation is also performed using VPA b-scan imaging (FIGS. 11C and 11D). The contrast from the lipid core and scattered lipid depositions are clearly observed when using 1730 nm excitation (FIG. 11C). Six times contrast reduction is observed when switching to 1210 nm excitation (FIG. 11D). This result is consistent with the phantom study shown in FIG. 11C. The VPA spectrum (see FIG. 11E) was taken at the position pointed by the red arrow. The spectrum matches the profile of CH₂ first overtone absorption.

### Selective VPA Imaging of Lipids and Proteins in the New Optical Window

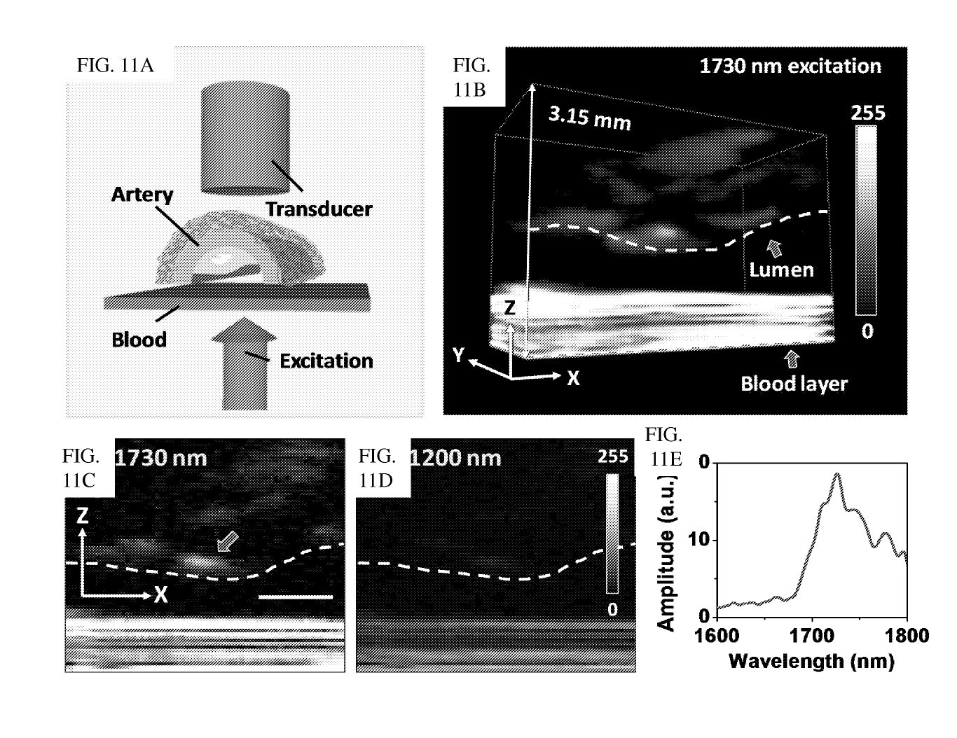

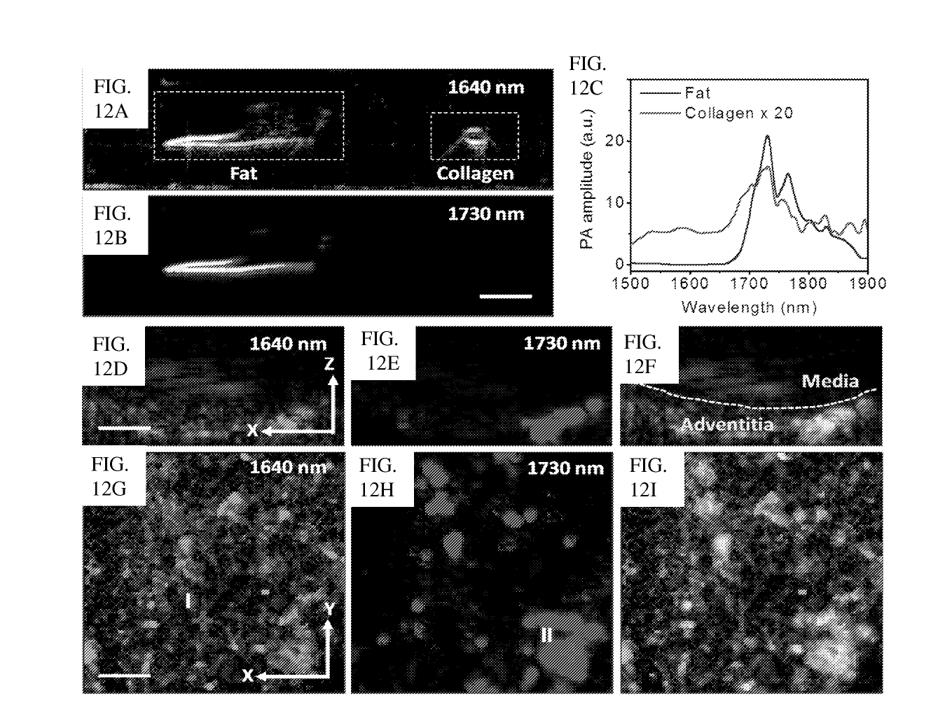

Bond-selective VPA imaging in biological samples can be achieved owing to the distinguishable spectral feature of CH₂ and CH₃ groups in first overtone region. To demonstrate this concept, a phantom which consisted butter fat (mainly lipid) and rat tail tendon (mainly type I collagen) was constructed. FIG. 12A shows the spectra of butter fat and rat tail tendon. The fat sample has a very high density of CH₂ group, therefore the spectrum shows a clear two-peak feature at 1730 and 1760 nm. For the spectrum of type I collagen (multiply by 20 in FIG. 12A), the spectral profile of CH₂ group is still visible and a shoulder appears at around 1700 nm which indicate the presents of CH₂ group. The result suggests that collagen sample has a higher CH₃/CH₂ ratio compared to fat sample. As can be observed, the contrast of collagen is higher than fat at spectral range of 1.5-1.65 μm owing to the spectral tail of CH₃ group. As a result, VPA imaging of the phantom sample was performed at both 1640 nm and 1730 nm (FIG. 12B and 12C). The result shows that lipid and protein can be differentiated using 1730 nm and 1640 nm excitation. As a further demonstration on biological sample, a 3D VPA imaging of artery adventitia was performed. The artery adventitia consist a large amount of type I collagen with vascular fat at the surrounding. The intact artery was placed in the glass bottom dish and stabilized by agarose-deuterium oxide gel. The contrast at 1640 nm, which attributes to the type I collagen, is different from the contrast at 1730 nm which comes from vascular fat. The different spectra profile at collagen abundant area and lipid abundant area confirms the capability of VPA imaging to differentiate the lipid and protein content. The results are depicted in FIGS. 12D-12I.

As discussed above, a Nd:YAG pumped optical parametric oscillator (OPO, Panther Ex Plus, Continuum) was utilized as the excitation source. The excitation module provides 10 Hz, 5 ns pulses laser with the wavelength range from 400 nm up to 2500 nm, covering both visible and near-infrared region. The near-infrared light, mostly produced at the idler beam from the OPO, was directed to an inverted microscope (IX71, Olympus) for spectroscopy and imaging purposes. The laser irradiation was then focused by an achromatic doublet lens (30 mm focal length, Thorlabs). A focused-type, 20 MHz ultrasound transducer with a 50% bandwidth (V317, Olympus NDT) was employed to detect the photoacoustic signal. A 30 dB low noise preamplifier (5682, Olympus NDT) and a receiver (5073PR-15-U, Olympus NDT) with adjustable gain were applied for receiving signal. The signal was then sent to a digitizer (USB-5133, National Instrument), record by PC via a LabVIEW (National Instrument) program.

The computer-controlled OPO system with automatic laser wavelength scanning enables the VPA spectroscopic study in a rapid way. The VPA spectra of water and deuterium oxide were taken by directly loading the sample to a glass bottom dish and focusing the laser beam to the glass-sample interface. The VPA spectrum of polyethylene was acquired when placing the polyethylene film to the glass-bottom dish and covering it with 2.5% agarose-deuterium oxide gel, since deuterium oxide has no significant absorption profile at the wavelength range we investigated. For the VPA spectra of 2,2,4-trimethylpentane, the sample was loaded into a glass tube of 1 mm inner diameter. The sample tube was then placed in a glass-bottom dish, and immersed in water. The midpoint of the tube was located within the focus of the transducer. The radiation beam was weakly focused and centered in the sample tube. The VPA signal was normalized according to the irradiation pulse energy at sample. For the 3 dimensional VPA imaging, a 2 dimensional scanning stage (ProScan H117, Prior) was employed for the raster scanning. The sample was embedded in 2.5% agarose-deuterium oxide gel to minimize the water absorption.

Image reconstruction.

The recorded signal waveforms were analyzed with a program on a MATLAB (MathWorks) platform. Hilbert transformation was performed to retrieve the envelope of the signal amplitude. The signals were reconstructed into a 3-D array for image reconstruction according to the locations coded in the time-resolved waveforms and the XY scanning pattern. The generated volumetric image renders sectional images, maximum amplitude projection (MAP) images, and 3-D images. The 2-D images were reconstructed under the MATLAB program, while 3-D images and movies were built via ImageJ and Voxx, respectively.

Monte Carlo simulation for evaluation of the effect of blood scattering and Absorption to the VPA Signal.

The Monte Carlo Simulation Was Performed To Calculate The excitation light attenuation by whole blood according to the software described in referance. The simulation is based on cylindrical coordinates. The separations between grid lines in z and r direction of cylindrical coordinate system were set as 5 μm and 40 μm, respectively. The grid elements numbers in r direction was set as 250, respectively. The simulation parameters of white matter tissue including absorption coefficient (μₐ), scattering coefficient (μₛ), scattering anisotropy parameter (g) and refractive index (n) are listed in Table 2 based on the reference.

The simulation was based on Gaussian beam with the waist w₀ (1/e² radius of the Gaussian beam), which is estimated based on following equation

$${w}_{0} = \frac{λ}{π \times N . A .} \qquad ( 4 )$$

where λ the wavelength of the light, N.A. is the numerical aperture of the Gaussian beam. In our case, the light was weakly focused by a lens doublet with 30 mm focus length. Since the photoacoustic signal which reaches the focal volume of ultrasound transducer (around 200 μm in radius) can be detected, only the photons reach the focal volume of ultrasound transducer was considered capable to generate signal. Therefore, the transparency of the irradiation at the focal area through the blood was calculated to estimate the excitation which reaches the sample.

TABLE 2

| | μₛ (cm⁻¹) | μₐ (cm⁻¹) | g | n |
|---|---|---|---|---|
| 1210 nm | 604 | 1.26 | 0.97 | 1.33 |
| 1730 nm | 414 | 6.40 | 0.95 | 1.33 |

Artery Samples from Ossabaw Porcine Model.

Pigs were fed excess calorie atherogenic diet, which was composed of 2% cholesterol, 20% kcal from fructose, and 43% kcal from hydrogenated soybean oil coconut oil, and lard. The genetic predisposition of Ossabaw pigs to obesity and metabolic syndrome promotes the development of atherosclerosis. Iliac arteries and the bifurcation of the internal and external carotids were harvested and then preserved in 10% phosphate-buffered formalin. Before imaging was performed, arteries were washed by phosphate-buffered saline and incised longitudinally for luminal imaging.

Those skilled in the art will recognize that numerous modifications can be made to the specific implementations described above. Therefore, the following claims are not to be limited to the specific embodiments illustrated and described above. The claims, as originally presented and as they may be amended, encompass variations, alternatives, modifications, improvements, equivalents, and substantial equivalents of the embodiments and teachings disclosed herein, including those that are presently unforeseen or unappreciated, and that, for example, may arise from applicants/patentees and others.

1. An imaging system, comprising:
a radiation source configured to output a signal that can non-invasively and selectively cause overtone excitation of molecules based on a predetermined chemical bond; and
an ultrasound detector configured to non-invasively detect an acoustic signal generated by vibrational energy caused by the selective overtone excitation of the molecules and further configured to convert the acoustic signal into an image.

2. The imaging system of claim 1, wherein the signal provided by the radiation source is configured to provide a label-free imaging of lipid-rich atherosclerotic plaques.

3. The imaging system of claim 1, wherein the signal provided by the radiation source is pulsed.

4. The imaging system of claim 1, wherein the signal provided by the radiation source is wavelength-tunable.

5. The imaging system of claim 1, wherein the signal provided by the radiation source is monochromatic.

6. The imaging system of claim 1, wherein the signal provided by the radiation source is pulsed, wavelength-tunable, and monochromatic.

7. The imaging system of claim 4, wherein the wavelength of the signal provided by the radiation source is adjusted to match the overtone vibrational frequency of the molecules at near-infrared region.

8. The imaging system of claim 6, wherein the wavelength of the signal provided by the radiation source is adjusted to match the overtone vibrational frequency of a molecule at near-infrared region.

9. The imaging system of claim 1, the radiation source comprising:
a laser source;
an optical parametric oscillator configured to receive a first signal from the laser source and output a second signal; and
an optical expander configured to receive the second signal and output a third signal.

10. The imaging system of claim 1, further comprising:
an energy sensor configured to measure energy of the third signal.

11. The imaging system of claim 10, wherein the energy sensor is configured to provide a feedback signal to the radiation source for fine-tuning the signal provided by the radiation source.

12. The imaging system of claim 9, wherein the third signal is a near infrared signal.

13. The imaging system of claim 1, the ultrasound detector further comprising:
a transducer configured to convert mechanical vibration received from tissue into an electrical signal.

14. The imaging system of claim 13, the ultrasound detector further comprising:
a data acquisition software for analyzing the electrical signal and providing a feedback signal to the radiation source for fine-tuning the signal provided by the radiation source.

15. The imaging system of claim 1, wherein the acoustic signal can be converted into an image from a depth of at least 1 mm.

16. The imaging system of claim 1, further comprising:
a catheter which includes a receiving device positioned near a tip of the catheter and configured to detect the acoustic signal.

17. The imaging system of claim 16, wherein the radiation source is positioned near the tip of the catheter.

18. A method for imaging biological tissue, comprising:
providing a radiation signal from a radiation source that can non-invasively and selectively cause overtone excitation of molecules based on a predetermined chemical bond;
receiving an acoustic signal generated by vibrational energy caused by the selective overtone excitation of the molecules; and
converting the acoustic signal to an image representative of a biological tissue targeted by the radiation signal.

19. The method of claim 18, wherein the radiation signal is configured to provide a label-free imaging of lipid-rich atherosclerotic plaques.

20. The method of claim 18, wherein the radiation signal is pulsed.

21. The method of claim 18, wherein the radiation signal is wavelength-tunable.

22. The method of claim 18, wherein the radiation signal is monochromatic.

23. The method of claim 18, wherein the radiation signal is pulsed, wavelength-tunable, and monochromatic.

24. The method of claim 21, wherein the wavelength of the radiation signal is adjusted to match the overtone vibrational frequency of the molecules at near-infrared region.

25. The method of claim 23, wherein the wavelength of the radiation signal is adjusted to match the overtone vibrational frequency of a molecule at near-infrared region.

26. The method of claim 18, further comprising:
sensing energy in the radiation signal;
providing a feedback signal to the radiation source; and
fine tuning the radiation source based on the feedback signal.

27. The method of claim 26, further comprising:
transducing mechanical vibration received from the biological tissue into an electrical signal.

28. The method of claim 27, further comprising:
analyzing the electrical signal and providing a feedback signal to the radiation source for fine-tuning the radiation signal.
